(19) United States
(12) Patent Application Publication
(10) Pub. No.: US 2020/0005941 A1
(43) Pub. Date: Jan. 2, 2020

(54) MEDICAL ADVERSE EVENT PREDICTION, REPORTING, AND PREVENTION

(71) Applicant: THE JOHNS HOPKINS UNIVERSITY, Baltimore, MD (US)

(72) Inventors: Suchi SARIA, Baltimore, MD (US); Hossein SOLEIMANI, Baltimore, MD (US)

(21) Appl. No.: 16/489,971

(22) PCT Filed: Mar. 1, 2018
(86) PCT No.: PCT/US2018/020394

§ 371 Date: Aug. 29, 2019

Related U.S. Application Data
(60) Provisional application No. 62/465,947, filed on Mar. 2, 2017.

(51) Int. Cl. G16H 50/20; G16H 10/20

(52) U.S. Cl. CPC G16H 50/20; G16H 10/20

(57) ABSTRACT

Disclosed are techniques for predicting, reporting, and preventing medical adverse events, such as septicemia. The techniques may be implemented in a client-server arrangement, where the clients are present on medical professionals' smart phone, for example. The disclosed techniques' ability to detect impending medical adverse events utilizes two innovations. First, some embodiments include a flexible and scalable joint model based upon sparse multiple-output Gaussian processes. Unlike state-of-the-art joint models, the disclosed model can explain highly challenging structure including non-Gaussian noise while scaling to large data. Second, some embodiments utilize an optimal policy for predicting events using the distribution of the event occurrence estimated by the joint model.

### RELATED APPLICATION

The present application claims priority to, and the benefit of, U.S. Provisional Patent Application No. 62/465,947 entitled, “Medical Adverse Event Prediction and Reporting” to Saria et al., filed on Mar. 2, 2017, which is hereby incorporated by reference in its entirety.

### FIELD

This disclosure relates generally to predicting, reporting, and preventing impending medical adverse events.

### BACKGROUND

Septicemia is the eleventh leading cause of death in the U.S. Mortality and length of stay decrease with timely treatment.

Missing data and noisy observations pose significant challenges for reliably predicting adverse medical events from irregularly sampled multivariate time series (longitudinal) data. Imputation methods, which are typically used for completing the data prior to event prediction, lack a principled mechanism to account for the uncertainty due to missingness.

### SUMMARY

According to various embodiments, a method of predicting an impending medical adverse event is disclosed. The method includes: obtaining a global plurality of test results, the global plurality of test results including, for each of a plurality of patients, and each of a plurality of test types, a plurality of patient test results obtained over a first time interval; scaling up, by at least one an electronic processor, a model of at least a portion of the global plurality of test results, such that a longitudinal event model including at least on random variable is obtained; determining, by at least one electronic processor, for each of the plurality of patients, and from the longitudinal event model, a hazard function including at least one random variable, where each hazard function indicates a chance that an adverse event occurs for a respective patient at a given time conditioned on information that the respective patient has not incurred an adverse event up until the given time; generating, by at least one electronic processor, for each of the plurality of patients, a joint model including the longitudinal event model and a time-to-event model generated from the hazard functions, each joint model indicating a chance of an adverse event occurring within a given time interval; obtaining, for a new patient, and each of a plurality of test types, a plurality of new patient test results obtained over a second time interval; applying, by at least one electronic processor, the joint model to the plurality of new patient test results obtained of the second time interval; obtaining, from the joint model, an indication that the new patient is likely to experience an impending medical adverse event within a third time interval; and sending an electronic message to a care provider of the new patient indicating that the new patient is likely to experience an impending medical adverse event.

Various optional features of the above embodiments include the following. The adverse event may be septicemia. The plurality of test types may include creatinine level. The sending may include sending a message to a mobile telephone of a care provider for the new patient. The longitudinal event model and the time-to-event model may be learned together. The testing phase may further include applying a detector to the joint model, where an output of the detector is confined to: yes, no, and abstain. The longitudinal event model may provide confidence intervals about a predicted test parameter level. The generating may include learning the longitudinal event model and the time-to-event model jointly. The scaling up may include applying a sparse variational inference technique to the model of at least a portion of the global plurality of test results. The scaling up may include applying one of: a scalable optimization based technique for inferring uncertainty about the global plurality of test results, a sampling based technique for inferring uncertainty about the global plurality of test results, a probabilistic method with scalable exact or approximate inference algorithms for inferring uncertainty about the global plurality of test results, or a multiple imputation based method for inferring uncertainty about the global plurality of test results.

According to various embodiments, a system for predicting an impending medical adverse event is disclosed. The system includes at least one mobile device and at least one electronic server computer communicatively coupled to at least one electronic processor and to the at least one mobile device, where the at least one electronic processor executes instructions to perform instructions including: obtaining a global plurality of test results, the global plurality of test results including, for each of a plurality of patients, and each of a plurality of test types, a plurality of patient test results obtained over a first time interval; scaling up, by at least one an electronic processor, a model of at least a portion of the global plurality of test results, such that a longitudinal event model including at least on random variable is obtained; determining, by at least one electronic processor, for each of the plurality of patients, and from the longitudinal event model, a hazard function including at least one random variable, where each hazard function indicates a chance that an adverse event occurs for a respective patient at a given time conditioned on information that the respective patient has not incurred an adverse event up until the given time; generating, by at least one electronic processor, for each of the plurality of patients, a joint model including the longitudinal event model and a time-to-event model generated from the hazard functions, each joint model indicating a chance of an adverse event occurring within a given time interval; obtaining, for a new patient, and each of a plurality of test types, a plurality of new patient test results obtained over a second time interval; applying, by at least one electronic processor, the joint model to the plurality of new patient test results obtained over the second time interval; obtaining, from the joint model, an indication that the new patient is likely to experience an impending medical adverse event within a third time interval; and sending an electronic message to the mobile device of the medical professional of the new patient indicating that the new patient is likely to experience an impending medical adverse event.

Various optional features of the above embodiments include the following. The adverse event may be septicemia. The plurality of test types may include creatinine level. The mobile device may include a mobile telephone of a care provider for the new patient. The longitudinal event model and the time-to-event model may be learned together. The testing phase may further include applying a detector to the joint model, where an output of the detector is confined to: yes, no, and abstain. The longitudinal event model may provide confidence intervals about a predicted test parameter level. The generating may include learning the longitudinal event model and the time-to-event model jointly. The scaling up may include applying a sparse variational inference technique to the model of at least a portion of the global plurality of test results. The scaling up may include applying one of: a scalable optimization based technique for inferring uncertainty about the global plurality of test results, a sampling based technique for inferring uncertainty about the global plurality of test results, a probabilistic method with scalable exact or approximate inference algorithms for inferring uncertainty about the global plurality of test results, or a multiple imputation based method for inferring uncertainty about the global plurality of test results.

### DESCRIPTION OF THE DRAWINGS

The accompanying drawings, which are incorporated in and constitute a part of this specification, illustrate implementations of the described technology. In the figures:

FIG. 1 presents diagrams illustrating observed longitudinal and time-to-event data as well as estimates from a joint model based in this data according to various embodiments;

FIG. 2 is an example algorithm for a robust event prediction policy according to various embodiments;

FIG. 3 is a schematic diagram illustrating three example decisions made using a policy according to the algorithm of FIG. 2, according to various embodiments;

FIG. 4 presents data from observed signals for a patient with septic shock and a patient with no observed shock, as well as estimated event probabilities conditioned on fit longitudinal data, according to various embodiments;

FIG. 5 illustrates Receiver Operating Characteristic (“ROC”) curves, as well as True Positive Rate (“TPR”) and False Positive Rate (“FPR”) curves according to various embodiments;

FIG. 6 is a mobile device screenshot of a patient status listing according to various embodiments;

FIG. 7 is a mobile device screenshot of a patient alert according to various embodiments;

FIG. 8 is a mobile device screenshot of an individual patient report according to various embodiments;

FIG. 9 is a mobile device screenshot of a treatment bundle according to various embodiments;

FIG. 10 is a flowchart of a method according to various implementations; and

FIG. 11 is a schematic diagram of a computer communication system suitable for implementing some embodiments of the invention.

### DETAILED DESCRIPTION

Reference will now be made in detail to example implementations, which are illustrated in the accompanying drawings. Where possible the same reference numbers will be used throughout the drawings to refer to the same or like parts.

Existing joint modeling techniques can be used for jointly modeling longitudinal and medical adverse event data and compute event probabilities conditioned on the longitudinal observations. These approaches, however, make strong parametric assumptions and do not easily scale to multivariate signals with many observations. Therefore, some embodiments include several innovations. First, some embodiments include a flexible and scalable joint model based upon sparse multiple-output Gaussian processes. Unlike state-of-the-art joint models, the disclosed model can explain highly challenging structure including non-Gaussian noise while scaling to large data. Second, some embodiments utilize an optimal policy for predicting events using the distribution of the event occurrence estimated by the joint model. The derived policy trades-off the cost of a delayed detection versus incorrect assessments and abstains from making decisions when the estimated event probability does not satisfy the derived confidence criteria. Experiments on a large dataset show that the proposed framework significantly outperforms state-of-the-art techniques in event prediction.

1. Introduction

Some embodiments at least partially solve the problem of predicting events from noisy, multivariate longitudinal data—repeated observations that are irregularly-sampled. As an example application, consider the challenge of reliably predicting impending medical adverse events, e.g., in the hospital. Many life-threatening adverse events such as sepsis and cardiac arrest are treatable if detected early. Towards this, one can leverage the vast number of signals—e.g., heart rate, respiratory rate, blood cell counts, creatinine—that are already recorded by clinicians over time to track an individual's health status. However, repeated observations for each signal are not recorded at regular intervals. Instead, the choice of when to record is driven by the clinician's index of suspicion. For example, if a past observation of the blood cell count suggests that the individual's health is deteriorating, they are likely to order the test more frequently leading to more frequent observations. Further, different tests may be ordered at different times leading to different patterns of “missingness” across different signals. Problems of similar nature arise in monitoring the health of data centers and predicting failures based on the longitudinal data of product and system usage statistics.

In statistics, the task of event prediction may be cast under the framework of time-to-event or survival analysis. Here, there are two main classes of approaches. In the first, the longitudinal and event data are modeled jointly and the conditional distribution of the event probability is obtained given the longitudinal data observed until a given time. Some prior art techniques, for example, posit a Linear Mixed-Effects (“LME”) model for the longitudinal data. The time-to-event data are linked to the longitudinal data via the LME parameters. Thus, given past longitudinal data at any time t, one can compute the conditional distribution for probability of occurrence of the event within any future interval A. Some techniques allow a more flexible model that makes fewer parametric assumptions: specifically, they fit a mixture of Gaussian Process but they focus on single time series. In general, state-of-the-art techniques for joint-modeling of longitudinal and event data require making strong parametric assumptions about the form of the longitudinal data in order to scale to multiple signals with many observations. This need for making strong parametric assumptions limits applicability to challenging time series (such as those addressed by some embodiments).

An alternative class of approaches uses two-stage modeling: features are computed from the longitudinal data and a separate time-to-event predictor is learned given the features. For signals that are irregularly sampled, the missing values are completed using imputation and point estimates of the features are extracted from the completed data for the time-to-event model. An issue with this latter class of approaches is that they have no principled means of accounting for uncertainty due to missingness. For example, features may be estimated more reliably in regions with dense observations compared to regions with very few measurements. But by ignoring uncertainty due to missingness, the resulting event predictor is more likely to trigger false or missed detections in regions with unreliable feature estimates.

Yet additional existing techniques treat event forecasting as a time series classification task. This includes transforming the event data into a sequence of binary labels, 1 if the event is likely to occur within a given horizon, and 0 otherwise. However, to binarize the event data, an operator selects a fixed horizon (Δ). Further, by doing so, valuable information about the precise timing of the event (e.g., information about whether the event occurs at the beginning or near the end of the horizon (Δ) may be lost. For prediction, a sliding window may be used for computing point estimates of the features by using imputation techniques to complete the data or by using model parameters from fitting a sophisticated probabilistic model to the time-series data. These methods suffer from similar shortcomings as the two-stage time-to-event analysis approaches described above: they do not fully leverage uncertainty due to missingness in the longitudinal data.

Thus, some embodiments address the following question: can uncertainty due to missingness in the longitudinal data be exploited to improve reliability of predicting future events? Embodiments are presented that answer the question in the affirmative and that a reliable event prediction framework comprising one or both of the following innovations.

First, a flexible Bayesian nonparametric model for jointly modeling the high-dimensional, multivariate longitudinal and time-to-event data is presented. This model may be used for computing the probability of occurrence of an event, H(Δ|y^{0:t},t), within any given horizon (t, t+Δ] conditioned on the longitudinal data y^{0:t} observed until t. Compared with existing state-of-the-art in joint modeling, this approach scales to large data without making strong parametric assumptions about the form of the longitudinal data. Specifically, there is no need to assume simple parametric models for the time series data. Multiple-output Gaussian Processes (GPs) are used to model the multivariate, longitudinal data. This accounts for non-trivial correlations across the time series while flexibly capturing structure within a series. Further, in order to facilitate scalable learning and inference, some embodiments include e a stochastic variational inference algorithm that leverages sparse-GP techniques. This reduces complexity of inference from O(N³D³) to O(NDM²), where N is the number of observations per signal, D is the number of signals, and M (<<N) is the number of inducing points, which are introduced to approximate the posterior distributions.

Second, a decision-theoretic approach to derive an optimal detector which uses the predicted event probability H (Δ|y^{0:t},t) and its associated uncertainty to trade-off the cost of a delayed detection versus the cost of making incorrect assessments is utilized.

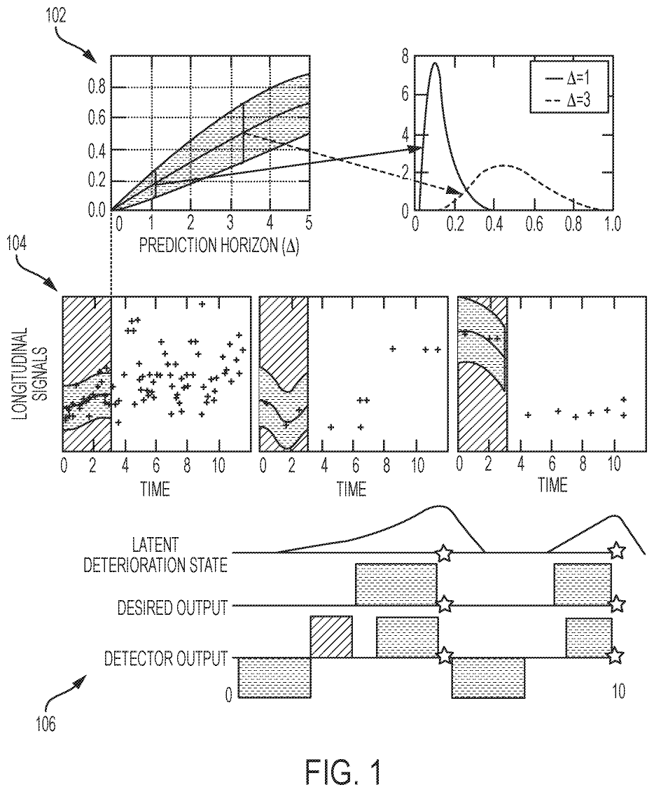

FIG. 1 presents diagrams illustrating observed longitudinal data 104 and time-to-event data 102, as well as estimates from a joint model based in this data according to various embodiments. As shown in the example detector output 106, the detector may choose to wait in order to avoid the cost of raising a false alarm. Others have explored other notions of reliable prediction. For instance, classification with abstention (or with rejection) has been studied before. Decision making in these methods are based on point-estimates of the features and the event probabilities. Others have considered reliable prediction in classification of segmented video frames each containing a single class. In these approaches, a goal is to determine the class label as early as possible.

2. Survival Analysis

This section presents survival analysis and joint models as used in some embodiments. In general, survival analysis references a class of statistical models developed for predicting and analyzing survival time: the remaining time until an event of interest happens. This includes, for instance, predicting time until a mechanical system fails or until a patient experiences a septic shock. The main focus of survival analysis as used herein is computing survival probability; i.e., the probability that each individual survives for a certain period of time given the information observed so far.

More formally, for each individual i, let Tᵢ ∈ [symbol] ³⁰ be a non-negative continuous random variable representing the occurrence time of an impending event. This random variable is characterized using a survival function, S(t)=Pr(T≥t); i.e., the probability that the individual survives up to time t. Given the survival function, it is possible to compute the probability density function

$$p ( t ) = - \frac{\partial }{\partial t} S ( t ) .$$

In survival analysis, this distribution is usually specified in terms of a hazard function, λ(t), which is defined as the instantaneous probability that the event happens conditioned on the information that the individual has survived up to time t; i.e.,

$$\begin{matrix}\begin{matrix}λ ( t ) {=}^{Δ} { lim}_{Δ \to 0} \frac{1}{Δ} Pr ( t < T \leq t + Δ \vert T \geq Δ ) \\ = \frac{P ( t )}{S ( t )} = - \frac{\partial }{\partial t} log S ( t ) .\end{matrix} & ( 1 )\end{matrix}$$

From Equation (1), S(t)=exp(−∫₀^{t}λ(s)ds) and p(t)=λ(t)exp(−∫₀^{t}λ(s)ds) may be easily computed.

In the special case where λ(t)=λ₀, where λ₀ is a constant, this distribution reduces to the exponential distribution with p(t)=λ₀ exp(λ₀t). In general, the hazard (risk) function may depend on some time-varying factors and individual specific features. A suitable parametric choice for hazard function for an individual who has survived up to time t is

λ(s; t)=λ₀(s; t)exp(α^{T}fᵢ^{0:t}), ∀s≥t, (2)

where fᵢ^{0:t} is a vector of features computed based on longitudinal observations up to time t, and α is a vector of free parameter which should be learned. Also, λ₀(s; t) is a baseline hazard function that specifies the natural evolution of the risk for all individuals independently of the individual-specific features. Typical parametric forms for λ₀(s; t) are piece-wise constant functions and λ(s; t)=exp(a+b(s−t)), t, where a and b are free parameters. Some embodiments utilize the latter form.

Given this hazard function, a quantity of interest in time-to-event models is event probability (failure probability), which may be defined as the probability that the event happens within the next Δ hours:

$$\begin{matrix}\begin{matrix}H ( Δ \vert {f}_{i}^{0 : t} , t ) {=}^{Δ} 1 - S ( t + Δ \vert {f}_{i}^{0 : t} , t ) = P ( T \leq t + Δ \vert {f}_{i}^{0 : t} , T \geq t ) \\ = 1 - exp ( {\int }_{t}^{t + Δ} λ ( s ; t ) ds )\end{matrix} & ( 3 )\end{matrix}$$

The event probability, H(Δ|fᵢ^{0:t}, t), is an important quantity in many applications. For instance, Equation (3) can be used as a risk score to prioritize patients in an intensive care unit and allocate more resources to those with greater risk of experiencing an adverse health event in the next Δ hours. Such applications may include dynamically updating failure probability as new observations become available over time.

Joint Modeling: The hazard function given by Equation (2) and the event probability of Equation (3) assume that the features fᵢ^{0:t} are deterministically computed from the longitudinal data up to time t. However, computing these features may be challenging in the setting of longitudinal data with missingness. In this setting, and according to some embodiments, probabilistic models are presented to jointly model the longitudinal and time-to-event data.

Let yᵢ^{0:t} be the longitudinal data up to time t for individual i. The longitudinal component models the time series yᵢ^{0:t} and estimates the distribution of the features conditioned on yᵢ^{0:t} i.e., p(fᵢ^{0:t}|yᵢ^{0:t}). Given this distribution, the time-to-event component models the survival data and estimates the event probability.

Note that because the features are random variables with distribution p(fᵢ^{0:t}|yᵢ^{0:t}), the event probability H(Δ|fᵢ^{0:t},t) is now a random quantity; i.e., every realization of the features drawn from p(fᵢ^{0:t}|yᵢ^{0:t}) computes a different estimate of the event probability. As a result, the random variable fᵢ^{0:t} induces a distribution on H(Δ|fᵢ^{0:t},t), i.e., p_{H}(H(Δ|fᵢ^{0:t},t)=h). This distribution may be obtained from the distribution p(fᵢ^{0:t}|yᵢ^{0:t}) using change-of-variable techniques.

Typically, expectation of H(Δ|fᵢ^{0:t},t) is computed for event prediction:

H̅(Δ, t) [symbol] ∫H(Δ|fᵢ^{0:t},t)p(fᵢ^{0:t}, |yᵢ^{0:t})dfᵢ^{0:t}=∫hpₕ(h)dh. (4)

However, some embodiments could also consider variance or quantiles of this distribution to quantify the uncertainty in the estimate of the event probability (see FIG. 1).

Learning: Joint models maximize the joint likelihood of the longitudinal and time-to-event data, Πᵢ₌₁^{I}p(yᵢ,Tᵢ), where p(yᵢ,Tᵢ)=∫p(yᵢ|fᵢ)p(Tᵢ|fᵢ)dfᵢ. In many practical situations, the exact event time for some individuals is not observed due to censoring. Some embodiments account for two types of censoring: right censoring and interval censoring. In right censoring, that the event did not happen before time Tᵣᵢ is known, but the exact time of the event is unknown. Similarly, in interval censoring, that the event happened within a time window, Tᵢ ∈[Tₗᵢ,Tᵣᵢ] is known. Given this partial information, the likelihood of the time-to-event component may be expressed as p(Tᵢ, δᵢ|fᵢ), with Tᵢ={Tᵢ,Tₗᵢ,Tᵣᵢ} and

$$p ( {T}_{i} , {δ}_{i} \vert {f}_{i} ) = \{ \begin{matrix}λ ( {T}_{i} ) S ( {T}_{i} ) , & if event censored ( {δ}_{i} = 0 ) \\ S ( {T}_{li} ) , & if right censored ( {δ}_{i} = 1 ) \\ S ( {T}_{li} ) - S ( {T}_{ri} ) , & if interval censored ( {δ}_{i} = 0 )\end{matrix} \qquad ( 5 )$$

where the explicit conditioning on fᵢ in λ(Tᵢ|fᵢ) and S(Tᵢ|fᵢ) is omitted for brevity and readability.

Note that the value of the hazard function (2) for each time s≥t depends on the history of the features f^{0:t}. Alternatively, the hazard rate is sometimes defined as a function of instantaneous features, i.e., λ(s)=λ₀(s)exp(α^{T}fᵢ(s)), ∀s. This definition is typically used when the focus of studies is retrospective analysis; i.e., to identify the association between different features and the event data. However, this approach may not be suitable for dynamic event prediction, which aims to predict failures well before the event occurs. In this approach, the probability of occurrence of the event within the (t, t+Δ] horizon involves computing

$$S ( t + Δ \vert {y}^{0 : t} ) = {E}_{[ {f}^{0 : t + Δ} \vert {y}^{0 : t} ]} exp ( - {\int }_{0}^{t + Δ} {λ}_{0} ( s ) exp ( {α}^{T} {f}_{i} ( s ) ) ds ) .$$

Obtaining the distribution of f^{0:t+Δ} conditioned on y^{0:t} is challenging, as it may include prospective prediction of the features for the (t, t+Δ] interval. Further, the expectation in S(t+Δ|y^{0:t}) may be computationally intractable. Instead, a dynamic training approach is typically taken which uses a hazard function defined in Equation (2). Here, the likelihood for each individual is evaluated at a series of grid points tᵢ₁≤tₜ₂ . . . ≤Tᵢ. At each training time point t, a new time-to-event random variable is defined with survival time Tᵢ−t with the hazard function λ(s;t), ∀s≥t. Intuitively, rather than modeling the instantaneous relation between the features and the event, this approach directly learns the association between the event probability and historical features. This is the approach used in some embodiments.

3. Joint Longitudinal and Time-to-Event Model

This section presents a framework to jointly model the longitudinal and time-to-event data. The probabilistic joint model includes two sub-models: a longitudinal sub-model and a time-to-event sub-model. Intuitively, the time-to-event model computes event probabilities conditioned on the features estimated in the longitudinal model. These two sub-models are learned together by maximizing the joint likelihood of the longitudinal and time-to-event data.

Let yᵢ^{0:t} be the observed longitudinal data for individual i until time t. A probabilistic joint modeling framework by maximizing the likelihood Πᵢp(Tᵢ, δᵢ, yᵢ^{0:t}), where Tᵢ and δᵢ are the time-to-event information defined in Section 2, is presented. Unless there is ambiguity, the superscripting with t is suppressed for readability henceforth.

The rest of this section introduces the two sub-models. This specifies the distribution p(Tᵢ,δᵢ,yᵢ^{0:t}). Next follows a description of how some embodiments jointly learn these longitudinal and time-to-event sub-models.

3.1 Longitudinal Sub-Model

Some embodiments use multiple-output Gaussian Processes (“GPs”) to model multivariate longitudinal data for each individual. GPs provide flexible priors over functions which can capture complicated patterns exhibited by clinical data. The longitudinal sub-model may be developed based on the known Linear Models of Co-regionalization (“LMC”) framework. LMC can capture correlations between different signals of each individual. This provides a mechanism to estimate sparse signals based on their correlations with more densely sampled signals.

Let y_{id}=y_{id}(t_{id})={y_{id}(t_{idn}), ∀n=1, 2, . . . , N_{id}} be the collection of N_{id} observations for signal d of individual i. Denote the collection of observations of D longitudinal signals of individual i by yᵢ={yᵢ₁, . . . , y_{iD}}. Assume, without loss of generality, that the data are Missing-at-Random (“MAR”); i.e., the missingness mechanism does not depend on unobserved factors. Under this assumption, process that caused missing data may be ignored, and parameters of the model may be inferred only based on the observed data.

Each signal y_{id}(t) may be expressed as:

y_{id}(t)=f_{id}(t)+ε_{id}(t),

f_{id}(t)=Σᵣ₌₁^{R}w_{idr}gᵢᵣ(t)+κ_{id}ν_{id}(t), (6)

where gᵢᵣ(t), ∀r=1, 2, . . . , R, are shared latent functions, v_{id}(t) is a signal-specific latent function, and w_{idr} and K_{id} are, respectively, the weighting coefficients of the shared and signal-specific terms.

Each shared latent function gᵢᵣ=gᵢᵣ(t_{id}) is a draw from a GP with mean 0 and covariance K_{N}_{_{id}}_{N}_{_{id}}^{(ir)}=Kᵢᵣ(t_{id}, t′_{id}); i.e., gᵢᵣ˜ [symbol] (0,K_{N}_{_{id}}_{N}_{_{id}}^{(ir)}) and gᵢᵣ⊥g_{i′r′}, ∀r≠r′, ∀i, i′. The parameters of this kernel are shared across different signals. The signal-specific function is generated from a GP whose kernel parameters are signal-specific: v_{id}˜ [symbol] (0, K_{N}_{_{id}}_{N}_{_{id}}^{(ir)}).

Some embodiments utilize the Matérn-1/2 kernel (e.g., as disclosed in C. E. Rasmussen and C. Williams, Gaussian Processes for Machine Learning, MIT Press, 2006) for each latent function. For shared latent functions, for instance,

$${K}_{ir} ( t , {t}^{′} ) = exp ( - \frac{1}{2} \frac{\vert t - {t}^{′} \vert}{{l}_{ir}} )$$

where Iᵢᵣ>0 is the length-scale of the kernel, and |t −t′| is the Euclidean distance between t and t′.

Assume, without loss of generality, that ε_{id}(t) is generated from a non-standardized Student's t-distribution with scale σ_{id} and three degrees of freedom, ε_{id}(t)˜T₃(0, σ_{id}). Some embodiments utilize Student's t-distribution because it has heavier tail than Gaussian distribution and is more robust against outliers.

Intuitively, this particular structure of the model posits that the patterns exhibited by the multivariate time-series of each individual can be described by two components: a low-dimensional function space shared among all signals and a signal-specific latent function. The shared component is the primary mechanism for learning the correlations among signals; signals that are more highly correlated give high weights to the same set of latent functions (i.e., w_{idr} and w′_{idr} are similar). Modeling correlations is natural in domains like health where deterioration in any single organ system is likely to affect multiple signals. Further, by modeling the correlations, the model can improve estimation when data are missing for a sparsely sampled signal based on the correlations with more frequently sampled signals.

Sharing kernel length-scale across individuals: The length-scale lᵢᵣ determines the rate at which the correlation between points decreases as a function of their distance in time. To capture common dynamic patterns and share statistical strength across individuals, some embodiments share the length-scale for each latent function across all individuals. However, especially in the dynamical setting where new observations become available over time, one length-scale may not be appropriate for all individuals with different length of observation. Experimentally, the inventors found that the kernel length-scale may be defined as a function of the maximum observation time for each individual:

lᵢᵣ=J(γᵣ log t̅ᵢ)+βᵣ), ∀r=1, 2, . . . , R, (7)

where t̅ᵢ=max_{d} maxₙ t_{idn} is the maximum observed time for individual i, and γᵣ and βᵣ are population-level parameters which may be estimated along with other model parameters. Thus, instead of sharing the same length-scale between individuals who may have different length of observations, share γᵣ and βᵣ. Using this function, two individuals with the same t̅ᵢ will have the same length-scale. Also, J: [symbol] → [symbol] is an appropriate mapping to obtain positive length-scale. Set J(x)=10+15000/(1+exp(−x)) to obtain lᵢᵣ ∈[10, 15010]; this prevents too small or too large length-scales. In experiments, the inventors set R=2 and initialized β and γ such that one kernel captured short-term changes in the shared latent functions and the other learned the long-term trends. Some embodiments initialize γᵣ=1, ∀r=1, 2, β₁=−12, and β₂=−16. After initialization, some embodiments learn these parameters along with other parameters of the model.

Similarly define kernels and length-scales for signal-specific latent functions,

$${K}_{id} ( t , {t}^{′} ) = exp ( - \frac{1}{2} \frac{\vert t - {t}^{′} \vert}{{l}_{id}} ) ,$$

with l_{id}=J(γ_{d} log t̅ᵢ+β_{d}), ∀d=1,2, . . . , D, where t̅ᵢ=maxₙ t_{idn}, and γ_{d} and β_{d} are free parameters.

Unless there is ambiguity, henceforth drop the index for individual i. Also, to simplify the notation, assume t_{id}=tᵢ, ∀d, and write K_{NN}^{(r)}Kᵢᵣ(tᵢ,t′ᵢ). Note that the observations from different signals need not be aligned for the learning algorithm.

3.2 Time-to-Event Sub-Model

The time-to-event sub-model computes the event probabilities conditioned on the features fᵢ^{0:t} which are estimated in the longitudinal sub-model. Specifically, given the predictions fᵢ^{0:t} for each individual i who has survived up to time t, define a dynamic hazard function for time s≤t:

$$\begin{matrix}λ ( s ; t ) = exp ( b + a ( s - t ) + {\overline{f}}_{i} ( t ) ) , ∀ s \geq t , where & ( 8 ) \\ {\overline{f}}_{i} ( t ) = {α}^{T} {\int }_{0}^{t} {ρ}_{c} ( {t}^{′} ; t ) {f}_{i} ( {t}^{′} ) {dt}^{′} , & ( 9 ) \\ {ρ}_{c} ( {t}^{′} ; t ) = c \frac{exp ( - c ( t - {t}^{′} )}{1 - exp ( - ct )} , ∀ {t}^{′} ∈ [ 0 , t ] , & ( 10 )\end{matrix}$$

and fᵢ(t′) [symbol] [fᵢ₁(t), . . . ,f_{iD}(t)]^{T}. Here, ρ_{c}(t′; t) is the weighting factor for the integral, and c≥0 is a free parameter. At any time t, ρ_{c}(t′; t) gives exponentially larger weight to most recent history of the feature trajectories; the parameter c controls the rate of the exponential weight. The relative weight given to most recent history increases by increasing c. Some embodiments also normalize ρ so that ∫₀^{t}ρ_{c}(t′;t)dt′=1, ∀t, c.

We can also write the hazard function in terms of the latent functions by substituting Equation (6) into Equation (8):

λ(s; t)=exp(b+a(s−t)+Σ_{d=1}^{D}κ′_{d}∫₀^{t}ρ_{c}(t′;t)νᵢ(t′)dt′+ρᵣ₌₁^{R}ω′ᵣ∫₀^{t}ρ_{c}(t′;t)dt′), (11)

where κ′_{d} [symbol] κ_{d}α_{d} and ω′ᵣ [symbol] Σ_{d=1}^{D}ω_{dr}α_{d}. Section 3.3, describes how to analytically compute the integrals of the latent functions in Equation (11). Given (11), at any point t, some embodiments compute the distribution of the event probability p_{H}(h). For a given realization of f̅, the event probability may be expressed as:

$$H ( Δ \vert \overline{f , t} ) = 1 - exp ( - λ ( t ; t ) \frac{1}{a} ( {e}^{a Δ} - 1 ) ) . \qquad ( 12 )$$

The hazard function defined in Equation (8) is based on linear features (i.e., exp(α^{T}∫₀^{t}ρ_{c}(t′; t)fᵢ(t′)dt′). Linear features are common in survival analysis because they are interpretable. In some embodiments, interpretable features are preferred over non-linear features that are challenging to interpret. Non-linear features can be incorporated within the disclosed framework.

3.3 Learning and Inference

This section discloses learning and inference for the proposed joint model. Some embodiments utilize models with global and local parameters. Global parameters, denoted by Θ₀, are the parameters of the time-to-event model (α, a, b, c) and the parameters defining the kernel length-scales (γᵣ , γ_{d}, βᵣ, β₀), i.e., Θ₀={α, a, b, c, γᵣ, γ_{d}, βᵣ, β₀}. Some embodiments update the local parameters for a minibatch of individuals independently, and use the resulting distributions to update the global parameter. Unlike classical stochastic variational inference procedures, such local updates are highly non-linear, and some embodiments make use of gradient-based optimization inside the loop.

3.3.1 Local Parameters

A bottleneck for inference is the use of robust sparse GPs in the longitudinal sub-model. Specifically, due to matrix inversion, even in the univariate longitudinal setting, GP inference scales cubically in the number of observations. To reduce this computational complexity, some embodiments utilize a learning algorithm based on the sparse variational approach. Also, the assumption of heavy-tailed noise makes the model robust to outliers, but this means that the usual conjugate relationship in GPs may be lost: the variational approach also allows approximation of the non-Gaussian posterior over the latent functions. The local parameters of the disclosed model, denoted by Θᵢ comprise the variational parameters controlling these Gaussian process approximations, noise-scale, and inter-process weights ω, κ. Point-estimates of these parameters may be made.

The disclosed model involves multiple GPs: for each individual, there are R latent functions gᵣ and D signal-specific functions v_{d}. In the variational approximation, each of these functions is assumed independent, without loss of generality, and controlled by some inducing input-response pairs Z, u, where Z are some pseudo-inputs (which are arranged on a regular grid) and u are the values of the process at these points. The variables u are given a variational distribution q(u)= [symbol] (m, S) which gives rise to a variational GP distribution, q(gᵣ)= [symbol] (μ_{g}_{ᵣ}, Σ_{g}_{ᵣ}), ∀r=1, . . . , R, where μᵣ_{ᵣ}=K_{NZ}^{(r)}K_{ZZ}^{(r)-1}mᵣ and Σg_{gr}=K_{NN}^{(r)}−K_{NZ}^{(r)}K_{ZZ}^{(r)-1} (I−SᵣK_{ZZ}^{(r)-1})K_{ZN}^{(r)},

where K_{NZ}^{(r)}=Kᵣ(t, Z). The variational distribution q(v_{d})= [symbol] (μ_{vd},Σ_{vd}), ∀d=1, . . . , D may similarly be obtained.

Since the functions of interest f_{d}, ∀d=1, 2, . . . , D, are given by linear combinations of these processes, the variational distribution q(f) is given by taking linear combinations of these GPs. Specifically:

q(f_{d})= [symbol] (μ_{d},Σ_{d}), (13)

where μ_{d}=Σᵣ₌₁^{R}ω_{d}_{ᵣ}, μ_{g}_{ᵣ}κ_{d}μ_{ν}_{_{d}} and Σ_{d}=Σᵣ₌₁^{R}ω_{dr}²Σ_{gr}+κ_{d}²Σ_{νd}. These variational distributions are used to compute the ELBO, which is used as an objective function in optimizing the variational parameters m, S.

For each individual, longitudinal data y time-to-event data Tᵢ, and censoring data δᵢ is given. Collecting these into [symbol] ᵢ, the likelihood function for an individual is p( [symbol] ᵢ|Θᵢ, Θ₀). Hereon, drop the individual subscript, i, and the explicit conditioning on Θᵢ and Θ₀ for readability. Given the GP approximations and using Jensen's inequality, obtain:

ELBOᵢ=log ∫p( [symbol] |Θ, f)p(f|u)p(u)dfdu (14)
≥E_{q(f)}[log(p(y|f))+log(p(T,δ|f))]−KL(q(u)∥p(u)),

where q(f)=E_{q(f)}p(f|u). Computing Equation (14) may utilize the fact that the time-to-event and longitudinal data are independent conditioned on f.

First consider computation of E_{q(f)} and log(p(y|f)). Since conditioned on f, the distribution of y factorizes over d, obtain E_{q(f)} log(p(y|f))=Σ_{d}E_{q(f}_{_{d}}₎ log(P(y_{d}|f_{d})) where q(f_{d}) is computed in Equation (13). Given the choice of the noise distribution, this expectation cannot be computed analytically. However, conditioned on f_{d}, log(p(y_{d}|f_{d})) also factorizes over all individual observations. Thus, this expectation reduces to a sum of several one-dimensional integrals, one for each observation, which may be approximated using Gauss-Hermite quadrature.

Next consider computation of E_{q(f)}log(p(T, δ|f). Unlike y, likelihood of the time-to-event sub-model does not factorize over d. Some embodiments take expectations of the terms involving the hazard function (11) which involves computing integral of latent functions over time. To this end, some embodiments make use of the following property:

Let f(t) be a Gaussian process with mean piₜ and kernel function K(t,t′). Then ∫₀^{T}ρ(t)f(t)dt is a Gaussian random variable with mean ∫₀^{T}ρ(t)μ(t)dt and variance ∫₀^{T}∫₀^{T}ρ(t)K(t,t′)ρ(t′)dtdt′.

Using this property, it follows that fᵢ(t)=α^{T}∫₀^{t}ρ_{c}(t′; t)fₜ(t′)dt′ is a Gaussian random variable with mean μᵢ^{(t)} and variance σᵢ²^{ₜ}, which may be computed analytically in closed form. Then compute E_{q(f)}log(p(T, δ|f) by replacing the likelihood function as defined in Equation (5) and following the dynamic approach for defining the hazard function described in Section 3.2. Expectation of the term related to interval censoring in the likelihood function is not available in closed form. Instead, compute Monte Carlo estimate of this term and use reparameterization techniques for computing gradients of this term with respect to model parameters.

Now, compute ELBO, in Equation (14). The KL term in Equation (14) is available in closed form.

3.3.2 Global Parameters

This section describes estimation of the global parameters Θ₀={α, a, b, c, γᵣ, γ_{d}, βᵣ, β₀}. The overall objective function for maximizing Θ₀ is: ELBO=Σᵢ^{I}ELBOᵢ where I is the total number of individuals. Since ELBO is additive over I terms, some embodiments can use stochastic gradient techniques. At each iteration of the algorithm, randomly choose a mini-batch of individuals and optimize ELBO with respect to their local parameters (as discussed in Section 3.3.1), keeping Θ₀ fixed. Then perform one step of stochastic gradient ascent based on the gradients computed on the mini-batch to update global parameters. Repeat this process until either relative change in global parameters is less than a threshold or maximum number of iterations is reached. Some embodiments use AdaGrad for stochastic gradient optimization.

Some embodiments utilize software that automatically computes gradients of the ELBO with respect to all variables and runs the learning algorithm in parallel on multiple processors.

4. Uncertainty Aware Event Prediction

The joint model developed in Section 3 computes the probability of occurrence of the event H(Δ|f̅, t) within any given horizon Δ. This section derives the optimal policy that uses this event probability and its associated uncertainty to detect occurrence of the event. The desired behavior for the detector is to wait to see more data and abstain from classifying when the estimated event probability is unreliable and the risk of incorrect classification is high. To obtain this policy, some embodiments take a decision theoretic approach.

At any given time, the detector takes one of the three possible actions: it makes a positive prediction (i.e., to predict that the event will occur within the next Δ hours), negative prediction (i.e., to determine that the event will not occur during the next Δ hours), or abstains (i.e., to not make any prediction). The detector decides between these actions by trading off the cost of incorrect classification against the penalty of abstention. Define a risk (cost) function by specifying a relative cost term associated with each type of possible error (false positive and false negative) or abstention. Then derive an optimal decision function (policy) by minimizing the specified risk function.

Specifically, for every individual i, given the observations up to time t, the aim is to determine whether the event will occur (ψᵢ=1) within the next Δ hours or not (ψᵢ=0). Hereon, again drop the i and t subscripts for brevity. Treat ψ as an unobserved Bernoulli random variable with probability Pr(ψ=1)=H(Δ|f̅, t). The joint model estimates this probability by computing the distribution pH(h). The distribution on H provides valuable information about the uncertainty around the estimate of Pr(ψ=1). The robust policy, presented next, uses this information to improve reliability of event predictions.

Denote the decision made by the detector by \hat{ψ}. The optimal policy chooses an action \hat{ψ} ∈{0, 1, a}, where a indicates abstention, and \hat{ψ}=0, 1, respectively, denote negative and positive prediction.

Specify the risk function by defining L₀₁ and L₁₀, respectively, as the cost terms associated with false positive (if ψ=0 and \hat{ψ}=1) and false negative (if ψ=1 and \hat{ψ}=0) errors and defining Lₐ as the cost of abstention (if \hat{ψ}=a). Conditioned on ψ, the overall risk function is

R(\hat{ψ};ψ)=1(\hat{ψ}=0)ψL₁₀+1(\hat{ψ}=1)(1−ψ)L₀₁+=1(\hat{ψ}=a)Lₐ, (15)

where the indicator function, 1(x), equals 1 or 0 according to whether the boolean variable x is true or false.

Since ψ is an unobserved random variable, instead of minimizing Equation (15), minimize the expected value of R(\hat{ψ}; ψ) with respect to the distribution of ψ, Pr(ψ=1)=H: i.e., R(\hat{ψ}; H)=1(\hat{ψ}=0)H L₁₀+1(\hat{ψ}=1) (1−H)L₀₁+1(\hat{ψ}=a)Lₐ. Because H is a random variable, the expected risk function R(\hat{ψ}; H) is also a random variable for every possible choice of \hat{ψ}. The distribution of R(\hat{ψ}; H) can be easily computed based on the distribution of H, pH(h).

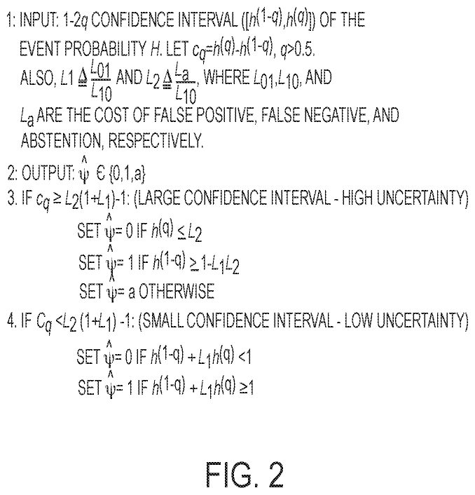

FIG. 2 is an example algorithm for a robust event prediction policy according to various embodiments. Obtain the robust policy by minimizing the quantiles of the risk distribution. Intuitively, by doing this, the maximum cost that could occur with a certain probability is minimized. For example, with probability 0.95, the cost under any choice of if) is less than R^{(0.95)}, the 95th quantile of the risk distribution R(\hat{ψ}; H).

Specifically, let h^{(q)} be the q-quantile of the distribution pH(h); i.e., f₀^{h(q)}pH(h)dh=q. Compute the q-quantile of the risk function, R^{(q)}(\hat{ψ}), for \hat{ψ}=0, 1, or α.

When \hat{ψ}=0, the q-qunatile of the risk function is L₁₀h^{(q)}. Similarly, for the case of \hat{ψ}=1, the q-quantile of the risk function is L₀₁h^{(1-q)}. Here, use the property that the q-quantile of the random variable 1−H is 1−h^{(1-q)}, where h^{(1-q)} is the (1−q)-quantile of H. Finally, q-quantile of the risk function is Lₐ in the case of abstention (\hat{ψ}=a). Obtain the q-quantile of the risk function:

R^{(q)}(\hat{ψ})=1(\hat{ψ}=0)h^{(q)}L₁₀+1(\hat{ψ}=1)(1−h^{(1-q)})L₀₁+1(\hat{ψ}=a)Lₐ. (16)

Minimize Equation (16) to compute the optimal policy. The optimal policy determines when to choose \hat{ψ}=0, 1, or a as a function of h^{(q)},h^{(1-q)}, and the cost terms L₀₁, L₁₀, and Lₐ. In particular, choose \hat{ψ}=0 when h(q) L₁₀≤(1−h^{(1-q)}L₀₁ and h^{(q)}L₁₀≤Lₐ. Because the optimal policy only depends on the relative cost terms, to simplify the notation, define

$${L}_{1} {=}^{Δ} \frac{{L}_{01}}{{L}_{10}} and {L}_{2} {=}^{Δ} \frac{{L}_{a}}{{L}_{10}} .$$

Further, assume without loss of generality that q>0.5 and define c_{q} [symbol] h^{(q)}−h^{(1-q)}. Here, c_{q} is the 1−2q confidence interval of H. Therefore, substituting L₁, L₂, and c_{q}, the condition for choosing \hat{ψ}=0 simplifies to h^{(q)}≤(1+c_{q})/(1+L₁) and h^{(q)}≤L₂.

Similarly obtain optimal conditions for choosing \hat{ψ}=1 or \hat{ψ}=a. The optimal decision rule is given as follows:

$$\begin{matrix}\hat{ψ} = \{ \begin{matrix}0 , & if {h}^{( q )} \leq \underline{τ} ( {c}_{q} ) , \\ 1 , & if {h}^{( q )} \geq \overline{τ} ( {c}_{q} ) , \\ a , & if \underline{τ} ( {c}_{q} ) < {h}^{( q )} < \overline{τ} ( {c}_{q} )\end{matrix} & ( 17 ) \\ where \underline{τ} ( {c}_{q} ) = min \{ {L}_{2} , \frac{1 + {c}_{q}}{1 + {L}_{1}} \} and & \\ \overline{τ} ( {c}_{q} ) = max \{ 1 + {c}_{q} - {L}_{1} {L}_{2} , \frac{1 + {c}_{q}}{1 + {L}_{1}} \} . & \end{matrix}$$

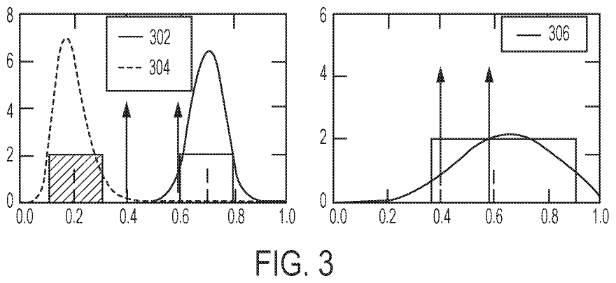

FIG. 3 is a schematic diagram illustrating three example decisions made using a policy according to the algorithm of FIG. 2, according to various embodiments. In particular, FIG. 3 illustrates three example decisions made using the policy described in FIG. 2 with L₁=1 and L₂=0.4. The shaded area is the confidence interval [h^{(1-q)}, h^{(q)}] for some choice of q for the three distributions, 302, 304, 306. The arrows at 0.4 and 0.6 are L₂ and 1−L₁L₂, respectively. All cases satisfy c_{q}≥L₂ (1+L₁)−1. The optimal decisions are \hat{ψ}=0 for 302, \hat{ψ}=1 for 304, and \hat{ψ}=a for 306.

The thresholds τ(c_{q}) and τ̅(c_{q}) in (17) can take two possible values depending on how c_{q} is compared to L₁ and L₂: in the special case that c_{q}>L₂(1+L₁)−1, the prediction may be made by comparing the confidence interval [h^{(1-q)}, h^{(q)}] against thresholds L₂ and 1−L₁L₂. In particular, if the entire confidence interval is below L₂ (i.e., if h^{(q)}<L₂ as shown in FIG. 3 at 302), declare \hat{ψ}=0. If the entire confidence interval is above 1−L₁L₂ (i.e., if h^{(1-q)}>1−L₁L₂ as shown in FIG. 3 at 304), predict \hat{ψ}=1. And if none of these conditions are met, the classifier abstains from making any decision (as shown in FIG. 3 at 306). In the case of c_{q}<L₂(1+L₁)−1 (i.e., the uncertainty level is below a threshold), \hat{ψ} is 0 or 1, respectively, if h^{(1-q)}+L₁h^{(q)} is less than or greater than 1. FIG. 2 summarizes this policy. The cost terms L₁, L₂, and q may be provided by the field experts based on their preferences for penalizing different types of error and their desired confidence level. Alternatively, a grid search on L₁, L₂, q may be performed, and the combination that achieves the desired performance with regard to specificity, sensitivity and the false alarm rates selected. In experiments, the inventors took the latter approach.

4.1. Special Case: Policy without Uncertainty Information

Imputation-based methods and other approaches that do not account for the uncertainty due to missingness can only compute point-estimates of the failure probability, H. In that case, the distribution over H can be thought of as a degenerate distribution with mass 1 on the point estimate of i.e., pH (h)=1(h−h₀), where h₀ is the point estimate of H. Here, because of the degenerate distribution, h^{(q)}=h^{(1-q)}=h₀ and c_{q}=0.

In this special case, the robust policy summarized in FIG. 2 reduces to the following simple case:

$$\hat{ψ} = \{ \begin{matrix}0 , & if {h}^{( q )} \leq \underline{τ} ( {c}_{q} ) , \\ 1 , & if {h}^{( q )} \geq \overline{τ} ( {c}_{q} ) \\ a , & if \underline{τ} ( {c}_{q} ) < {h}^{( q )} < \overline{τ} ( {c}_{q} )\end{matrix} \qquad ( 18 )$$

As an example, consider the case that L₁=1. Here, if the relative cost of abstention is L₂≤0.5, then τ̅=τ=0.5, which is the policy for a binary classification with no abstention and a threshold equal to 0.5. Alternatively, when L₂<0.5, the abstention interval is [L₂, 1−L₂]. In this case, the classifier chooses to abstain when the event probability L₂<h₀<1−L₂ (i.e., when h₀ is close to the boundary).

4.1.1. Comparison with the Robust Policy with Uncertainty

Both the robust policy of Equation (17) and its special case of Equation (18) are based on comparing a statistic with an interval, i.e., h^{(q)} with the interval [τ(c_{q}), τ̅(c_{q})] in the case of Equation (17), and h₀ with the interval [τ,τ̅] in the case of Equation (18).

An important distinction between these two cases is that, under the policy of Equation (18), the abstention region only depends on L₁ and L₂ which are the same for all individuals, but under the robust policy of Equation (17), the length of the abstention region is max{0, c_{q}−(L₂(1+L₁)−1)}. That is, the abstention region adapts to each individual based on the length of the confidence interval for the estimate of H. The abstention interval is larger in cases where the classifier is uncertain about the estimate of H. This helps to prevent incorrect predictions. For instance, consider example 306 in FIG. 3. Here the expected value h₀ (dashed line) is greater than τ̅ but its confidence interval (shaded box) is relatively large. Suppose this is a negative sample, making a decision based on h₀ (policy of Equation (18)) will result in a false positive error. In order to abstain on this individual under the policy of Equation (18), the abstention interval should be very large. But because the abstention interval is the same for all individuals, making the interval too large leads to abstaining on many other individuals on whom the classifier may be correct. Under the robust policy, however, the abstention interval may be adjusted for each individual based on the confidence interval of H. In this particular case, for instance, the resulting abstention interval is large (because of large c_{q}), and therefore, the false positive prediction is avoided.

5. Experimenatal Results

The inventors evaluated the proposed framework on the task of predicting when patients in the hospital are at high risk for septic shock—a life-threatening adverse event. Currently, clinicians have only rudimentary tools for real-time, automated prediction for the risk of shock. These tools suffer from high false alert rates. Early identification gives clinicians an opportunity to investigate and provide timely remedial treatments.

5.1. Data

The inventors used the MIMIC-II Clinical Database, a publicly available database, consisting of clinical data collected from patients admitted to a hospital (the Beth Israel Deaconess Medical Center in Boston). To annotate the data, the inventors used the definitions for septic shock described in K. E. Henry et al., “A targeted real-time early warning score (TREWScore) for septic shock,” Science translational medicine, vol. 7, no. 299, p. 299ra122, 2015. Censoring is a common issue in this dataset: patients for high-risk of septic shock can receive treatments that delay or prevent septic shock. In these cases, their true event time (i.e. event under no treatment) is censored or unobserved. Some embodiments treat patients who received treatment and then developed septic shock as interval-censored because the exact time of shock onset could be at any time between the time of treatment and the observed shock onset time. Patients who never developed septic shock after receiving treatment are treated as right-censored. For these patients, the exact shock onset time could have been at any point after the treatment.

The inventors modeled the following 10 longitudinal streams: heartrate (“HR”), systolic blood pressure (“SBP”), urine output, Blood Urea Nitrogen (“BUN”), creatinine (“CR”), Glasgow coma score (“GCS”), blood pH as measured by an arterial line (“Arterial pH”), respiratory rate (“RR”), partial pressure of arterial oxygen (“PaO2”), and white blood cell count (“WBC”). These are the clinical signals used for identifying sepsis. In addition, the inventors also included the following static features that were shown to be highly predictive: time since first antibiotics, time since organ failure, and status of chronic liver disease, chronic heart failure, and diabetes.

The inventors randomly selected 3151 patients with at least two measurements from each signal. Because the original dataset was highly imbalanced, the inventors included all patients who experienced septic shock and have at least two observations per signal and sub-sampled patients with no observed shock to construct a relatively balanced dataset. The inventors randomly divided the patients into the training and test set. The training set consists of 2363 patients, including 287 patients with observed septic shock and 2076 event-free patients. Further, of the patients in the training set, 279 received treatment for sepsis, 166 of which later developed septic shock (therefore, they are interval censored); the remaining 113 patients are right censored. The test set included of 788 patients, 101 with observed shock and 687 event-free patients.

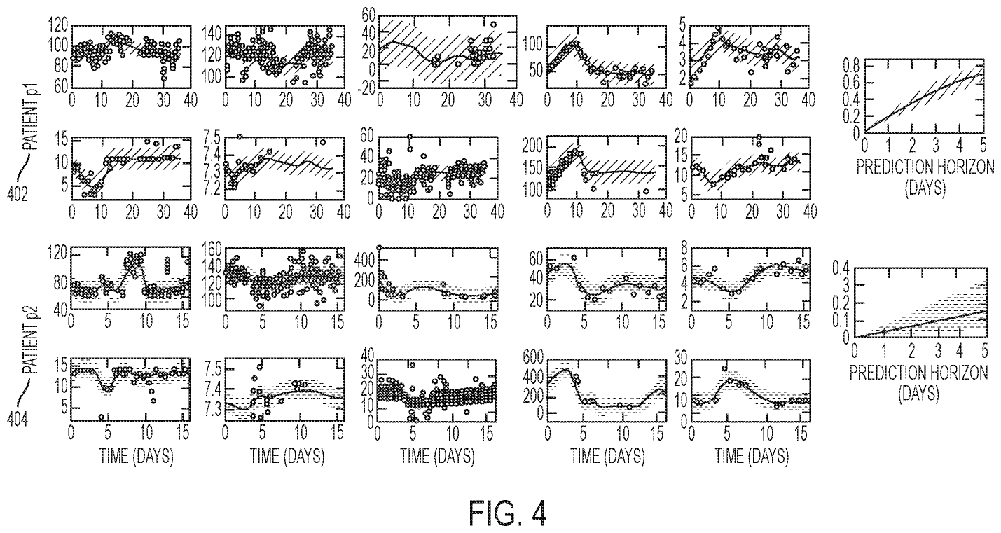

There are two challenging aspects of this data. First, individual patients have as many as 2500 observations per signal. This is several orders of magnitude larger than the size of data that existing state-of-the-art joint models can handle. Second, as shown in FIG. 4, these signals have challenging properties: non-Gaussian noise, some are sampled more frequently than others, the sampling rate varies widely even within a given signal, and individual signals contain structure at multiple scales.

5.2. Baselines

To understand the benefits of the proposed model, compare it with the following commonly used alternatives. 1. Because the original dataset is highly imbalanced, the inventors included all patients who experienced septic shock and have at least two observations per signal and sub-sampled patients with no observed shock to construct a relatively balanced dataset.

1) MoGP: For the first baseline, the inventors implement a two-stage survival analysis approach for modeling the longitudinal and time-to-event data. Specifically, the inventors fit a MoGP, which provides highly flexible fits for imputing the missing data. State-of-the-art performance for modeling physiologic data using multivariate GP-based models is possible. But, as previously discussed (see Section 3), their inference scales cubically in the number of recordings; thus, making it impossible to fit to a dataset contemplated herein. Here, the inventors use the GP approximations described in Section 3 for learning and inference. The inventors use the mean predictions from the fitted MoGP to compute features for the hazard function of Equation (8). Using this baseline, the inventors assess the extent to which a robust policy—that accounts for uncertainty due to the missing longitudinal data in estimating event probabilitiescontributes to improving prediction performance.

2) Logistic Regression: For the second baseline, the inventors use a time-series classification approach. Recordings from each time series signal are binned into four-hour windows; for bins with multiple measurements, the inventors use the average value. For bins with missing values, the inventors use covariate-dependent (age and weight) regression imputation. Binned values from 10 consecutive windows for all signals are used as features in a logistic regression (LR) classifier for event prediction. L2 regularization is used for learning the LR model; the regularization weight is selected using 2-fold cross-validation on the training data.

3) SVM: For the third baseline, the inventors replace the LR with a Support Vector Machine (“SVM”) to experiment with a more flexible classifier. The inventors use the RBF kernel and determine hyperparameters using two-fold cross-validation on the training data.

A final baseline to consider is a state-of-the-art joint model. As discussed before, existing joint-modeling methods require positing parametric functions for the longitudinal data: the inventors preliminary experiments using polynomial functions give very poor fits, which is not surprising given the complexity of the clinical data (see, e.g., FIG. 4). As a result, the inventors omit this baseline.

All of the baseline methods provide a point-estimate of the event probability at any given time. Thus, they use the special case of the robust policy with no uncertainty (the policy of Equation (18)) for event prediction.

Evaluation: The inventors computed the event probability and made predictions with Δ=12 hours prediction horizon. In order to avoid reporting bias from patients with longer hospital stays, for the purpose of evaluation, the inventors consider predictions at five equally spaced time points over the three day interval ending one hour prior to the time of shock onset or censoring. For the remaining, the inventors evaluate predictions during the last three days of their hospital stay.

For evaluation, all predictions are treated independently. Report performance of the classifiers as a function of the decision rate, which is the number of instances on which the classifier choose to make a decision; i.e., (Σᵢ1(\hat{ψ}ᵢ≠a))/(Σᵢ1). Perform a grid search on the relative cost terms L₁, L₂, and q (for the robust policy) and recorded population true positive rate (TPR), population false positive rate (FPR), and false alarm rate (FAR). These are TPR=(Σᵢ1(\hat{ψ}ᵢ=1, \hat{ψ}ᵢ=1))/(Σᵢ1(\hat{ψ}ᵢ=1)), FPR=(Σₜ1(\hat{ψ}ᵢ=1, \hat{ψ}ᵢ=0))/(Σᵢ1(\hat{ψ}ᵢ=0)), and FAR=(Σᵢ1(\hat{ψ}ᵢ=1, \hat{ψ}ᵢ=0))/(Σᵢ=1(\hat{ψ}ᵢ=1)).

To determine statistical significance of the results, the inventors perform non-parametric bootstrap on the test set with boot-strap sample size 20, and report the average and standard deviation of the performance criteria.

5.3. Numerical Results

FIG. 4 presents data from observed signals for a patient with septic shock and a patient with no observed shock, as well as estimated event probabilities conditioned on fit longitudinal data, according to various embodiments. Data from 10 signals (dots) and longitudinal fit (solid line) along with their confidence intervals (shaded area) for two patients, 402 patient p1 with septic shock and 404 patient p2 with no observed shock. On the right, the inventors show the estimated event probability for the following five day period conditioned on the longitudinal data for each patient shown on the left.

First, qualitatively investigate the ability of the proposed model—from hereon referred to as J-LTM—to model the longitudinal data and estimate the event probability. In FIG. 4, the fit achieved by J-LTM on all 10 signals for two patients is shown: a patient with septic shock (patient p1) and a patient who did not experience shock (patient p2). Note that HR, SBP, and respiratory rate (RR) are densely sampled; other signals like the arterial pH, urine output, and PaO2 are missing for long periods of time (e.g., there are no arterial pH and PaO2 recordings between days 15 and 31 for patient p1). Despite the complexity of their physiologic data, J-LTM can fit the data well. J-LTM captures correlations across signals. For instance, the respiratory rate for patient p2 decreases at around day four. The decrease in RR slows down the blood gas exchange, which in turn causes PaO2 to decrease since less oxygen is being breathed in. The decrease in RR also causes CO2 to build up in the blood, which results in decreased arterial pH. Also, the decrease in arterial pH corresponds to increased acidity level which causes mental status (GCS) to deteriorate. These correlations can be used to obtain a more reliable estimate of the event probability. Also note that J-LTM is robust against outliers. For instance, one measurement of arterial pH for patient p1 on day 5 is significantly greater than the other measurements from the same signal within the same day. Further, this sudden increase is not reflected in any other signal. Therefore, this single observation appears to be an outlier and may not be indicative of any change in the risk of developing septic shock. As a result (and partly due to the heavy-tailed noise model), J-LTM predictions for arterial pH on that day are not affected by this single outlier.

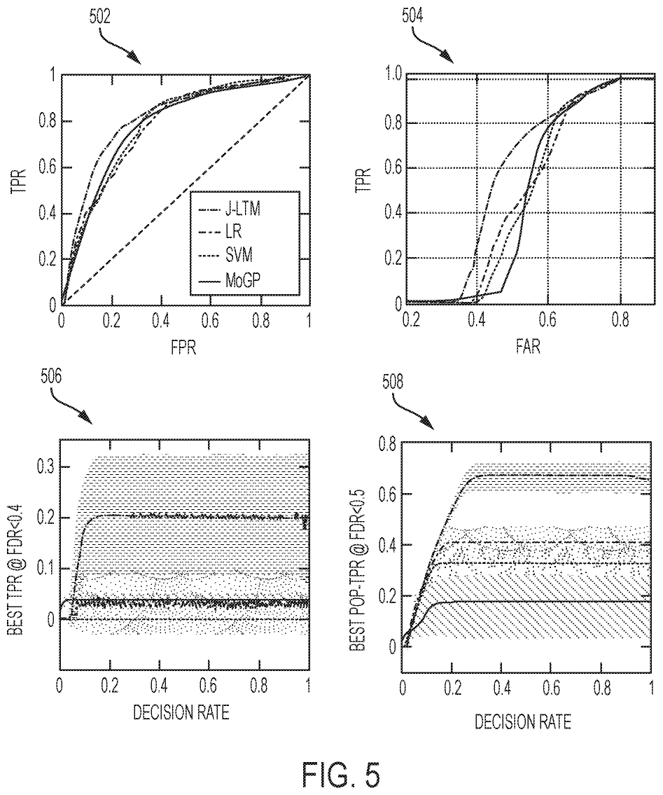

FIG. 5 illustrates Receiver Operating Characteristic (“ROC”) curves, as well as True Positive Rate (“TPR”) and False Positive Rate (“FPR”) curves according to various embodiments. As shown, FIG. 5 depicts ROC curves 502, Maximum TPR obtained at each FAR level 504, the best TPR achieved at any decision rate fixing FAR<0.4 506 and he best TPR achieved at any decision rate fixing FAR<0.5 508.

Next, quantitatively evaluate performance of J-LTM. The ROC curves (TPR vs. FPR) for J-LTM and the baseline methods (LR, SVM, and MoGP) are depicted in FIG. 5 at 502. To plot the ROC curve for each method, grid search on the relative cost terms L₁ and L₂ and q (for the robust policy) were performed, and the obtained FPR and TPR pairs recorded. J-LTM achieves an AUC of 0.82 (±0.02) and outperforms LR, SVM, and MoGP with AUCs 0.78 (±0.02), 0.79 (±0.02), and 0.78 (±0.02), respectively. As shown in FIG. 5 at 502, the increased TPR for J-LTM compared to the baseline methods primarily occurs for FPRs ranging from 0.1-0.5, the range most relevant for practical use. In particular, at FPR=0.15, J-LTM recovers 72% (±6) of the positive patients in the population. At the same FPR, TPR for LR, SVM, and MoGP are, respectively, 0.57 (±0.04), 0.58 (±0.05), and 0.61 (±0.05). It is worth noting that to do a fair comparison, the TPR and FPR rates shown in FIG. 5 at 502 are computed with respect to the population rather than the subset of instances where each method chooses to alert.

Further, FIG. 5 at 502 compares performance using the TPR and FPR but does not make explicit the number of false alerts. A performance criterion for alerting systems is the ratio of false alarms (FAR). Every positive prediction by the classifier may initiate attendance and investigation by the clinicians. Therefore, a high false alarm rate increases the workload of the clinicians and causes alarm fatigue. An ideal classifier detects patients with septic shock (high TPR) with few false alarms (low FAR). FIG. 5 at 504 plots the maximum TPR obtained at each FAR level for J-LTM and the baselines. At any TPR, the FAR for J-LTM is significantly lower than that of all baselines. In particular, in the range of TPR from 0.6 to 0.8, J-LTM shows 6% to 16% improvement in FAR over the next best baseline. From a practical standpoint, 16% reductions in the FAR can amount to many hours saved daily.

To elaborate on this comparison further, TPR and FAR for each method as a function of the number of decisions made (i.e., at 1, all models choose to make a decision for every instance) is examined. At a given decision rate, each model may abstain on a different subset of patients. FIGS. 5 at 506 and 508 depict the best TPR achieved at any given decision rate for two different settings of the maximum FAR. In FIG. 5 at 506, for example, at every abstention rate, the best TPR achieved for every model with the false alarm rate of less than 40% is plotted. J-LTM achieves significantly higher TPR than baseline methods at all decision rates. In other words, at any given decision rate, J-LTM is able to more correctly identify the subset of instances on whom it can make predictions. Similar plots are shown in FIG. 5 at 508: the maximum TPR with FAR<0.5 for J-LTM over all decision rates is 0.66 (±0.05). This is significantly greater than the best TPR at the same FAR level for LR, 0.41 (±0.06), SVM, 0.33 (±0.05), and MoGP, 0.18 (±0.14). A natural question to ask is whether the reported TPRs are good enough for practical use. The best standard-of-care tools implement the LR baseline without abstention. This corresponds to the performance of LR in FIG. 5 at 506 and 508 at the decision rate of 1. As shown, the gain in TPR achieved by J-LTM are large for both FAR settings.

6. Reporting Techniques and User Interface

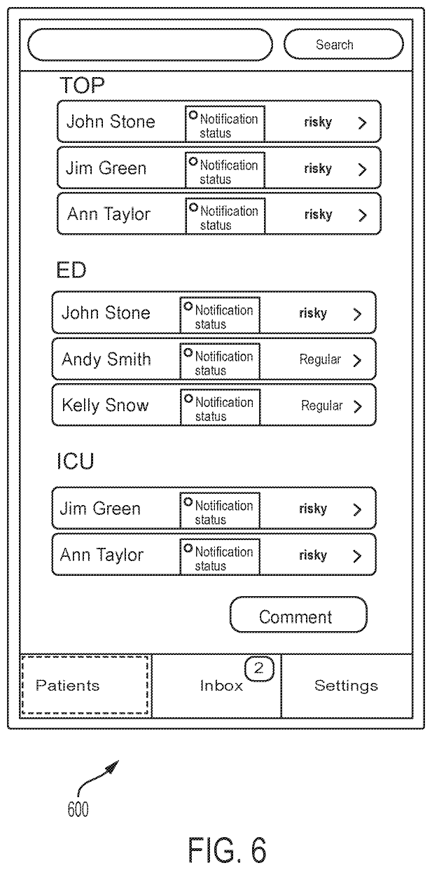

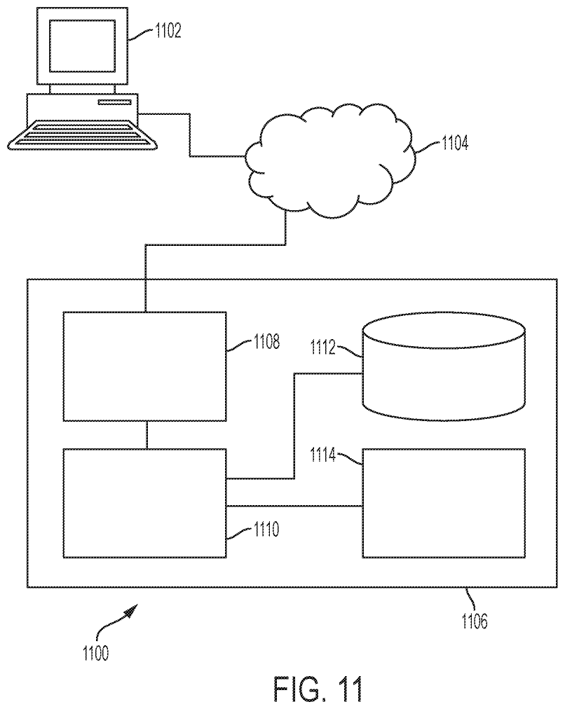

FIGS. 6-9 are example screenshots suitable for a user device that provides a user interface and patient reports. Such a user device may be implemented as user computer 1102 of FIG. 11, for example. In use, such a user device may be carried by a doctor or other medical professional. The user device may be used to enter empirical data, such as patient test results, into the system of some embodiments. Further, the user device may provide patient reports, and, if an adverse event is predicted, alerts.

FIG. 6 is a mobile device screenshot 600 of a patient status listing according to various embodiments. Screenshot 600 includes sections reflecting patient statuses for patients that are most at risk for a medical adverse event, patients that are in the emergency department, and patients that are in the intensive care unit. Entries for patients that are likely to experience an impending medical adverse event as determined by embodiments (e.g., the detector makes a positive prediction for a respective patient, H(Δ|f̅,t) exceeds some threshold such as 20% for some time interval Δ such as two hours, or the patient's TREWScore exceeds some threshold) are marked as “risky” or otherwise highlighted.

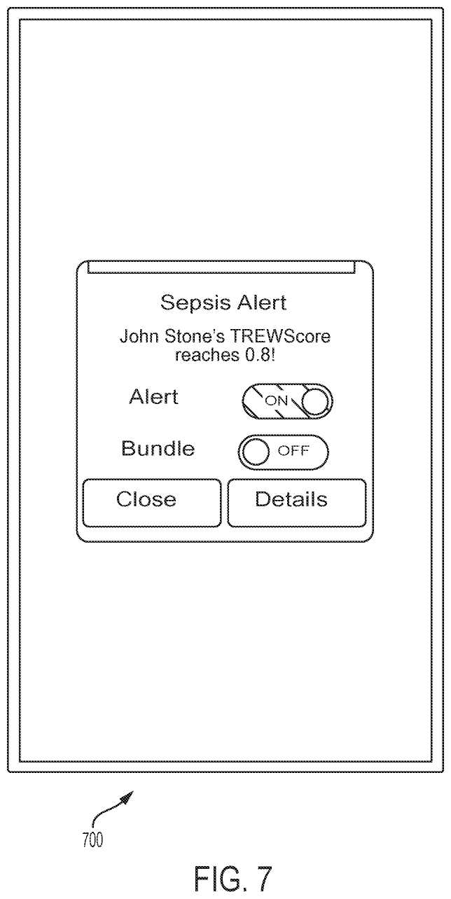

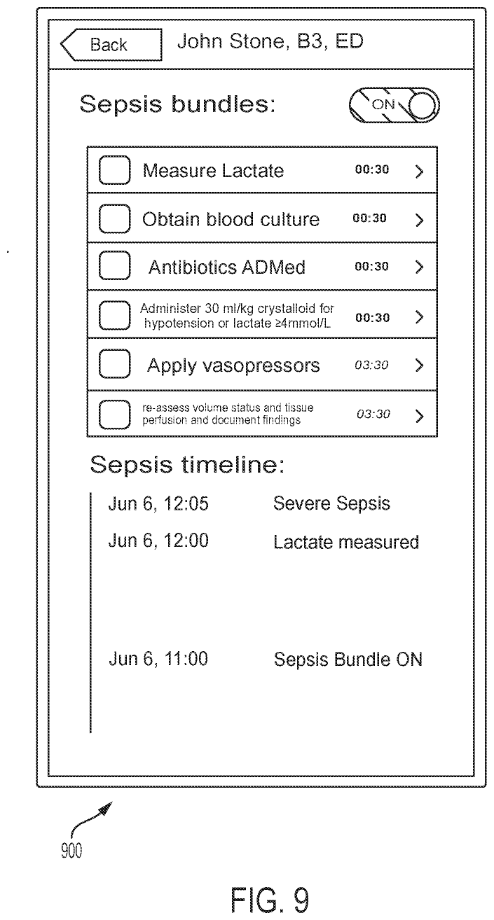

FIG. 7 is a mobile device screenshot 700 of a patient alert according to various embodiments. The user device may display an alert, possibly accompanied by a sound and/or haptic report, when a patient is determined to be at tisk for a medical adverse event according to an embodiment (e.g., the detector makes a positive prediction for a respective patient, H(Δ|f̅, t) exceeds some threshold such as 20% for some time interval Δ such as two hours, or the patient's TREWScore exceeds some threshold). The alert may specify the patient and include basic information, such as the patient's TREWScore. The alert may provide the medical professional with the ability to turn on a treatment bundle, described in detail below in reference to FIG. 9

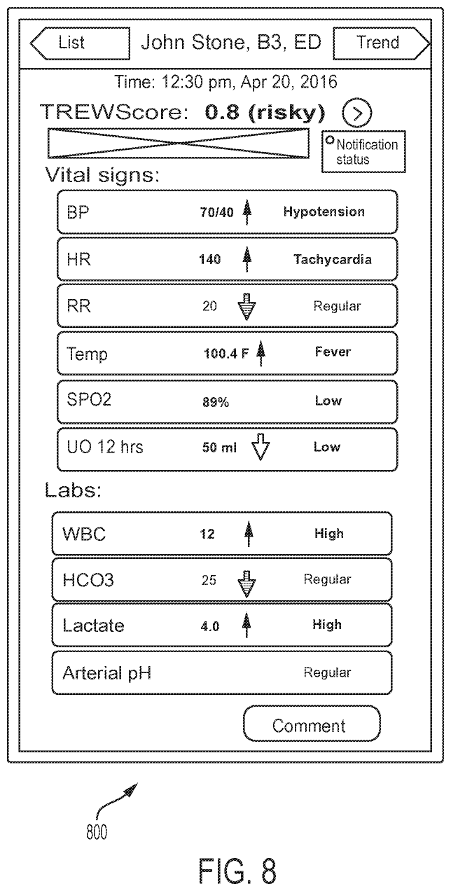

FIG. 8 is a mobile device screenshot 800 of an individual patient report according to various embodiments. The individual patient report includes a depiction of risk for the patient, e.g., the patient's TREWScore. The report may include any or all of the patient's most recent vital signs and lab reports. In general, any of the longitudinal data types may be represented and set forth.

FIG. 9 is a mobile device screenshot 900 of a treatment bundle according to various embodiments. The treatment bundle specifies a set of labs to be administered and therapeutic actions to be taken to thwart a medical adverse event. When activated, the treatment bundle provides alerts to the medical professional (and others on the team) to administer a lab or take a therapeutic action.

7. Conclusion

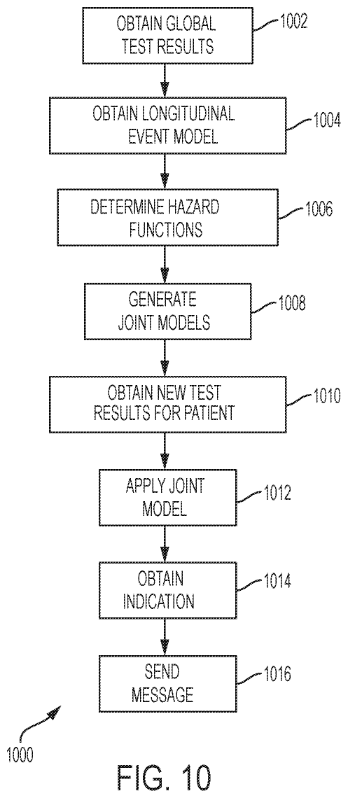

FIG. 10 is a flowchart of a method 1000 according to various embodiments. Method 1000 may be performed by a system such as system 1100 of FIG. 11.

At block 1002, method 1000 obtains a global plurality of test results, the global plurality of test results including, for each of a plurality of patients, and each of a plurality of test types, a plurality of patient test results. The actions of this block are described herein, e.g., in reference to the training set of patient records. The global plurality of test results may include over 100,000 test results.

At block 1004, method 1000 scales up a model of at least a portion of the global plurality of test results to produce a longitudinal event model. The actions of this block are as disclosed herein, e.g., in Section 3.1.

At block 1006, method 1000 determines, for each of a plurality of patients, and from the longitudinal event model, a hazard function. The actions of this block are disclosed herein, e.g., in Section 3.2.

At block 1008, method 1000 generates a joint model. The actions of this block are disclosed herein, e.g., in Section 3.3

At block 1010, method 1000 obtains, for each of a plurality of test types, a plurality of new patient test results for a patient. The actions of this block are disclosed throughout this document.

At block 1012, method 1000 applies the joint model for the new patient to the new patient test results. The actions of this block are disclosed herein, e.g., in Section 4.

At block 1014, method 1000 obtains an indication that the new patient is likely to experience an impending medical adverse event. The actions of this block are disclosed herein, e.g., in Section 4.

At block 1016, method 1000 sends a message to a medical professional indicating that the new patient is likely to experience a medical adverse event. The actions of this block are disclosed herein, e.g., in Section 6.

FIG. 11 is a schematic diagram of a computer communication system suitable for implementing some embodiments of the invention. System 1100 may be based around an electronic hardware internet server computer 1106, which may be communicatively coupled to network 1104. Network 1104 may be an intranet, a wide area network, the internet, a wireless data network, or another network. Server computer 1106 includes network interface 1108 to affect the communicative coupling to network 1104. Network interface 1108 may include a physical network interface, such as a network adapter or antenna, the latter for wireless communications. Server computer 1106 may be a special-purpose computer, adapted for reliability and high-bandwidth communications. Thus, server computer 1106 may be embodied in a cluster of individual hardware server computers, for example. Alternately, or in addition, server computer 1106 may include redundant power supplies. Persistent memory 1112 may be in a Redundant Array of Inexpensive Disk drives (RAID) configuration for added reliability, and volatile memory 1114 may be or include Error-Correcting Code (ECC) memory hardware devices. Server computer 1106 further includes one or more electronic processors 1110, which may be multi-core processors suitable for handling large amounts of information. Electronic processors 1110 are communicatively coupled to persistent memory 1112, and may execute instructions stored thereon to effectuate the techniques disclosed herein, e.g., method 1000 as shown and described in reference to FIG. 10. Electronic processors 1110 are also communicatively coupled to volatile memory 1114.

Server computer 1106 communicates with user computer 1102 via network 1104. User computer 1102 may be a mobile or immobile computing device. Thus, user computer 1102 may be a smart phone, tablet, laptop, or desktop computer. For wireless communication, user computer 1102 may be communicatively coupled to server computer 1106 via a wireless protocol, such as WiFi or related standards. User computer 1102 may be a medical professional's mobile device, which sends and receives information as shown and described herein, particularly in reference to FIGS. 6-9.

In sum, a probabilistic framework for improving reliability of event prediction by incorporating uncertainty due to missingness in the longitudinal data is disclosed. The approach comprised several innovations. First, a flexible Bayesian nonparametric model for jointly modeling high-dimensional, continuous-valued longitudinal and event time data is presented. In order to facilitate scaling to large datasets, a stochastic variational inference algorithm that leveraged sparse-GP techniques is used; this significantly reduces complexity of inference for joint-modeling from O(N³D³) to O(NDM²). Compared to state-of-the-art in joint modeling, the disclosed approach scales to datasets that are several order of magnitude larger without compromising on model expressiveness. The use of a joint-model enabled computation of the event probabilities conditioned on irregularly sampled longitudinal data. Second, a policy for event prediction that incorporates the uncertainty associated with the event probability to abstain from making decisions when the alert is likely to be incorrect is disclosed. On an important and challenging task of predicting impending in-hospital adverse events, the inventors have demonstrated that the disclosed model can scale to time-series with many measurements per patient, estimate good fits, and significantly improve event prediction performance over state-of-the-art alternatives.

Certain embodiments can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

While the invention has been described with reference to the exemplary embodiments thereof, those skilled in the art will be able to make various modifications to the described embodiments without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

What is claimed is:

1. A method of predicting an impending medical adverse event, the method comprising:
obtaining a global plurality of test results, the global plurality of test results comprising, for each of a plurality of patients, and each of a plurality of test types, a plurality of patient test results obtained over a first time interval;
scaling up, by at least one an electronic processor, a model of at least a portion of the global plurality of test results, whereby a longitudinal event model comprising at least on random variable is obtained;
determining, by at least one electronic processor, for each of the plurality of patients, and from the longitudinal event model, a hazard function comprising at least one random variable, wherein each hazard function indicates a chance that an adverse event occurs for a respective patient at a given time conditioned on information that the respective patient has not incurred an adverse event up until the given time;
generating, by at least one electronic processor, for each of the plurality of patients, a joint model comprising the longitudinal event model and a time-to-event model generated from the hazard functions, each joint model indicating a chance of an adverse event occurring within a given time interval;
obtaining, for a new patient, and each of a plurality of test types, a plurality of new patient test results obtained over a second time interval;
applying, by at least one electronic processor, the joint model to the plurality of new patient test results obtained of the second time interval;
obtaining, from the joint model, an indication that the new patient is likely to experience an impending medical adverse event within a third time interval; and
sending an electronic message to a care provider of the new patient indicating that the new patient is likely to experience an impending medical adverse event.

2. The method of claim 1, wherein the medical adverse event is septicemia.

3. The method of claim 1, wherein the plurality of test types include creatinine level.

4. The method of claim 1, wherein the sending comprises sending a message to a mobile telephone of a care provider for the new patient.

5. The method of claim 1, wherein the longitudinal event model and the time-to-event model are learned together.

6. The method of claim 1, further comprising applying a detector to the joint model, wherein an output of the detector is confined to: yes, no, and abstain.

7. The method of claim 1, wherein the longitudinal event model provides confidence intervals about a predicted test parameter level.

8. The method of claim 1, wherein the generating comprises learning the longitudinal event model and the time-to-event model jointly.

9. The method of claim 1, wherein the scaling up comprises applying a sparse variational inference technique to the model of at least a portion of the global plurality of test results.

10. The method of claim 1, wherein the scaling up comprises applying one of:
a scalable optimization based technique for inferring uncertainty about the global plurality of test results,
a sampling based technique for inferring uncertainty about the global plurality of test results,
a probabilistic method with scalable exact or approximate inference algorithms for inferring uncertainty about the global plurality of test results, or
a multiple imputation based method for inferring uncertainty about the global plurality of test results.

11. A system for predicting an impending medical adverse event, the system comprising at least one mobile device and at least one electronic server computer communicatively coupled to at least one electronic processor and to the at least one mobile device, wherein the at least one electronic processor executes instructions to perform operations comprising:
obtaining a global plurality of test results, the global plurality of test results comprising, for each of a plurality of patients, and each of a plurality of test types, a plurality of patient test results obtained over a first time interval;
scaling up, by at least one an electronic processor, a model of at least a portion of the global plurality of test results, whereby a longitudinal event model comprising at least on random variable is obtained;
determining, by at least one electronic processor, for each of the plurality of patients, and from the longitudinal event model, a hazard function comprising at least one random variable, wherein each hazard function indicates a chance that an adverse event occurs for a respective patient at a given time conditioned on information that the respective patient has not incurred an adverse event up until the given time;
generating, by at least one electronic processor, for each of the plurality of patients, a joint model comprising the longitudinal event model and a time-to-event model generated from the hazard functions, each joint model indicating a chance of an adverse event occurring within a given time interval;
obtaining, for a new patient, and each of a plurality of test types, a plurality of new patient test results obtained over a second time interval;
applying, by at least one electronic processor, the joint model to the plurality of new patient test results obtained over the second time interval;
obtaining, from the joint model, an indication that the new patient is likely to experience an impending medical adverse event within a third time interval; and
sending an electronic message to the mobile device indicating that the new patient is likely to experience an impending medical adverse event.

12. The system of claim 11, wherein the medical adverse event is septicemia.

13. The system of claim 11, wherein the plurality of test types include creatinine level.

14. The system of claim 11, wherein the mobile device comprises a mobile telephone of a care provider for the new patient.

15. The system of claim 11, wherein the longitudinal event model and the time-to-event model are learned together.

16. The system of claim 11, wherein the operations further comprise applying a detector to the joint model, wherein an output of the detector is confined to: yes, no, and abstain.

17. The system of claim 11, wherein the longitudinal event model provides confidence intervals about a predicted test parameter level.

18. The system of claim 11, wherein the generating comprises learning the longitudinal event model and the time-to-event model jointly.

19. The system of claim 11, wherein the scaling up comprises applying a sparse variational inference technique to the model of at least a portion of the global plurality of test results.

20. The system of claim 11, wherein the scaling up comprises applying one of:
a scalable optimization based technique for inferring uncertainty about the global plurality of test results,
a sampling based technique for inferring uncertainty about the global plurality of test results,
a probabilistic method with scalable exact or approximate inference algorithms for inferring uncertainty about the global plurality of test results, or
a multiple imputation based method for inferring uncertainty about the global plurality of test results.
